(19) United States
(12) Patent Application Publication
(10) Pub. No.: US 2020/0383371 A1
(43) Pub. Date: Dec. 10, 2020

(54) Smokable herb composition and product

(71) Applicant: Paul E. Mendell, Castle Rock, CO (US)

(72) Inventor: Paul E. Mendell, Castle Rock, CO (US)

(21) Appl. No.: 16/871,319

(22) Filed: May 11, 2020

Related U.S. Application Data
(60) Provisional application No. 62/845,864, filed on May 9, 2019.

(51) Int. Cl. A24B 15/16; A61K 31/05; A61K 36/185

(52) U.S. Cl. CPC A24B 15/16; A61K 36/185; A61K 31/05

(57) ABSTRACT

Smokable compositions and products comprising herbs and/or corn silk and a substantially THC-free composition are provided, as are methods of manufacture and use thereof. Compositions of the present invention may be suitable for use as delivery devices for therapeutic compounds and/or as cigarette substitutes or other smoking cessation aids.

### CROSS-REFERENCE TO RELATED APPLICATION

This application claims the benefit of priority of U.S. Provisional Patent Application 62/845,864, filed 9 May 2019, the entirety of which is incorporated herein by reference.

### FIELD OF THE INVENTION

The present invention relates generally to smokable herb, biomass materials such as corn silk, spirulina, or chlorella which are similar to cigarettes, and specifically to smokable herb products that do not contain hemp or tetrahydrocannabinol (THC) or similar compounds, as well as methods of manufacture and use thereof.

### BACKGROUND OF THE INVENTION

Tetrahydrocannabinol (THC) is the principal psychoactive constituent of cannabis. Under United States law, hemp products may contain no more than 0.3% THC. This limitation presents a challenge to the production and sale of products that may contain other constituents of cannabis/hemp plants, such as cannabidiol (CBD), because it can be difficult to produce extracts or constituents of hemp plants that contain 0.3% THC or less under a wide range of conditions because the testing criteria is based on dry weight of material and humidity and thus moisture content changes with changing conditions (e.g. dry conditions in which the moisture content of a hemp product is reduced).

CBD may have various therapeutic uses, including in the treatment of epilepsy and other neurological disorders. It is thus desirable to provide CBD in the form of products that may legally be produced and sold under U.S. law, i.e. as hemp products containing less than 0.3% THC, and especially desirable to provide smokable products, as these may additionally be used as substitutes and/or cessation aids for other smokable products that may be illegal (e.g. cannabis) or more hazardous (e.g. cigarettes) than hemp products.

Both spirulina and chlorella are biomass materials produced from cyanobacteria and algae, respectively. They are relatively high in protein and lower in carbohydrates. Because of the lower carbohydrates the combustion products associated will be substantially less than other smokable plant materials, for example tobacco. Both spirulina and chlorella are low in natural resins compared with tobacco and thus would result in lower tar which is known to be carcinogenic. Other carcinogenic compounds like polycyclic aromatic hydrocarbons (PAHs) would be substantially lower from combustion products of spirulina and chlorella.

There is thus a need in the art for smokable herb products similar to cigarettes, and specifically to smokable herb products that do not contain hemp or tetrahydrocannabinol (THC) or similar compounds.

### SUMMARY OF THE INVENTION

In aspects of the present disclosure, a smokable product comprises a composition of one or more smokable herbs, or plant material such as corn silk, spirulina, or chlorella; and a substantially THC-free composition.

In embodiments, the composition of one or more smokable herbs may comprise at least one herb selected from the group consisting of Althaea officinalis (marshmallow), Amaranthus dubius, Arctostaphylos uva-ursi (bearberry), Argemone mexicana, plants of the genus Arnica, Artemisia vulgaris (mugwort), plants of the family Asteraceae (chamomile), Brassica oleracea (cabbage), Calea zacatechichi, Canavalia maritima (baybean), plants of the genus Cannabis, Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), plants of the genus Lavandula (lavender), Lactuca virosa (lettuce opium), plants in the family Lamiaceae (mint) including but not limited to plants of the genuses Salvia (sage) and Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), species of the genus Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (both known as wireweed), Silene capensis, Syzygium aromaticum (clove), Tagetes lucida (Mexican tarragon), Tarchonanthus camphoratus, Turnera diffusa (damiana), Tussilago farfara (coltsfoot), species of the genus Verbascum (mullein), and Zornia latifolia (maconha brava).

In embodiments, the substantially THC-free composition may comprise any one or more compounds commonly found in or derived from plants of the genus Cannabis. The substantially THC-free composition may, but need not, be, or be derived from, a distillate obtained from plants of the genus Cannabis.

In embodiments, the substantially THC-free composition may comprise at least one cannabinoid selected from the group consisting of a cannabichromene-type (CBC) cannabinoid, a cannabichromanone-type (CBCN) cannabinoid, a cannabidiol-type (CBD) cannabinoid, a cannabielsoin-type (CBE) cannabinoid, a cannabigerol-type (CBG) cannabinoid, a cannabicyclol-type (CBL) cannabinoid, a cannabinol-type (CBN) cannabinoid, a cannabinodiol-type (CBND) cannabinoid, a cannabicitran-type or cannabitriol-type (CBT) cannabinoid, and an isocannabinoid.

In embodiments, the substantially THC-free composition may comprise at least one terpene or terpenoid selected from the group consisting of endo-borneol, δ-carene, bornyl acetate, α-ylangene, α-copaene, aromadendrene, eremophilene, longifolene, β-guaiene, valencene, β-bisabolene, γ-cadinene, β-selinene, neophytadiene, ferruginol, aristolone, β-amyrin, oleanane, ketoursene, α-amyrin, an iridoids, an iridoid glycoside, a steroid, a hemiterpenoid, an acyclic monoterpene, a monocyclic monoterpene, a bicyclic monoterpene, an acyclic monoterpenoid, a monocyclic monoterpenoid, a bicyclic monoterpenoid, a sesquiterpenoid, a diterpenoid, a sesterterpenoid, a non-steroidal triterpenoid, a sesquarterpene, a sesquarterpenoid, a carotene, a xanthophyll, a polyterpenoid, a norisoprenoid, and an activated isoprene.

In embodiments, the smokable product may comprise no more than 0.3 wt % THC.

In embodiments, the smokable product may be useful as a smoking cessation aid.

In aspects of the present disclosure, a method for making a smokable product comprises dissolving a substantially THC-free composition in a liquid solvent to form a substantially THC-free solution; applying the substantially THC-free solution to a composition of one or more smokable herbs; and evaporating the liquid solvent.

In embodiments, the substantially THC-free composition may comprise any one or more compounds commonly found in or derived from plants of the genus Cannabis. The substantially THC-free composition may, but need not, be, or be derived from, a distillate obtained from plants of the genus Cannabis.

In embodiments, the substantially THC-free composition may comprise at least one cannabinoid selected from the group consisting of a cannabichromene-type (CBC) cannabinoid, a cannabichromanone-type (CBCN) cannabinoid, a cannabidiol-type (CBD) cannabinoid, a cannabielsoin-type (CBE) cannabinoid, a cannabigerol-type (CBG) cannabinoid, a cannabicyclol-type (CBL) cannabinoid, a cannabinol-type (CBN) cannabinoid, a cannabinodiol-type (CBND) cannabinoid, a cannabicitran-type or cannabitriol-type (CBT) cannabinoid, and an isocannabinoid.

In embodiments, the substantially THC-free composition may comprise at least one terpene or terpenoid selected from the group consisting of endo-borneol, δ-carene, bornyl acetate, α-ylangene, α-copaene, aromadendrene, eremophilene, longifolene, β-guaiene, valencene, β-bisabolene, γ-cadinene, β-selinene, neophytadiene, ferruginol, aristolone, β-amyrin, oleanane, ketoursene, α-amyrin, an iridoids, an iridoid glycoside, a steroid, a hemiterpenoid, an acyclic monoterpene, a monocyclic monoterpene, a bicyclic monoterpene, an acyclic monoterpenoid, a monocyclic monoterpenoid, a bicyclic monoterpenoid, a sesquiterpenoid, a diterpenoid, a sesterterpenoid, a non-steroidal triterpenoid, a sesquarterpene, a sesquarterpenoid, a carotene, a xanthophyll, a polyterpenoid, a norisoprenoid, and an activated isoprene.

In embodiments, the liquid solvent may be an organic solvent. The organic solvent may, but need not, comprise an alcohol. The alcohol may, but need not, be ethanol.

In embodiments, the composition of one or more smokable herbs may comprise at least one herb selected from the group consisting of Althaea officinalis (marshmallow), Amaranthus dubius, Arctostaphylos uva-ursi (bearberry), Argemone mexicana, plants of the genus Arnica, Artemisia vulgaris (mugwort), plants of the family Asteraceae (chamomile), Brassica oleracea (cabbage), Calea zacatechichi, Canavalia maritima (baybean), plants of the genus Cannabis, Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), plants of the genus Lavandula (lavender), Lactuca virosa (lettuce opium), plants in the family Lamiaceae (mint) including but not limited to plants of the genuses Salvia (sage) and Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), species of the genus Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (both known as wireweed), Silene capensis, Syzygium aromaticum (clove), Tagetes lucida (Mexican tarragon), Tarchonanthus camphoratus, Turnera diffusa (damiana), Tussilago farfara (coltsfoot), species of the genus Verbascum (mullein), and Zornia latifolia (maconha brava).

In embodiments, the smokable product may comprise no more than 0.3 wt % THC.

In embodiments, the smokable product may be useful as a smoking cessation aid.

In aspects of the present disclosure, a method for aiding a subject in smoking cessation comprises providing to the subject a smoking cessation composition, wherein the smoking cessation composition comprises a plant material infused with at least one of nicotine, a cannabinoid, a terpene, and a terpenoid.

These and other advantages will be apparent from the disclosure of the aspects, embodiments, and configurations contained herein.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Unless otherwise noted, all component or composition levels are in reference to the active portion of that component or composition and are exclusive of impurities, for example, residual solvents or by-products, which may be present in commercially available sources of such components or compositions.

All percentages and ratios are calculated by total composition weight, unless indicated otherwise.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### DETAILED DESCRIPTION OF THE INVENTION

As used herein, the term “bio-incompatible” refers to a substance that is carcinogenic, toxic, or otherwise harmful to a human or animal, and/or to compositions of matter containing such substances. By analogy, as used herein, the term “bio-compatible” refers to a substance that is not carcinogenic, toxic, or otherwise harmful to a human or animal, and/or to compositions of matter that are substantially free of bio-incompatible substances.

As used herein, the term “smokable” refers to a substance that is, or may be, burned for the purpose of inhalation of the resulting smoke by a user. Generally, smokable substances are those whose smoke of combustion is intended to be inhaled, and subsequently tasted and/or absorbed into the bloodstream, by a person. Smokable substances include those that are smoked for any recreational and/or therapeutic purpose.

The present invention provides methods and systems for smokable herb products that do not contain hemp or tetrahydrocannabinol (THC) or similar compounds. In many embodiments, the smokable herb products obtained may comprise less than 0.3% THC and may therefore be legally manufactured and sold in the United States as hemp products, and may have uses including but not limited to neurological treatment devices and smoking cessation aids.

In embodiments of methods of the present invention, a substantially THC-free composition, usually a distillate obtained from cannabis/hemp plants, is provided. The substantially THC-free composition may comprise any one or more compounds other than THC commonly found in or derived from hemp plants, including, by way of non-limiting example, cannabichromene-type (CBC) cannabinoids, e.g. (±)-cannabichromene (CBC-C), (±)-cannabichromenic acid A (CBCA-C₅ A), (±)-cannabichromevarin (CBCV-C₃), and (±)-cannabichromevarinic acid A (CBCVA-C₃ A); cannabichromanone-type (CBCN) cannabinoids, e.g. cannabichromanone (CBCN-C), cannabichromanone-C₃ (CBCN-C₃), and cannabicoumaronone (CBCON-C₅); cannabidiol-type (CBD) cannabinoids, e.g. (−)-cannabidiol (CBD-C₅), cannabidiol monomethyl ether (CBDM-C₅), cannabidiol-C₄ (CBD-C₄), (−)-cannabidivarin (CBDV-C₃), cannabidiorcol (CBD-C₁), cannabidiolic acid (CBDA-C₅), and cannabidivarinic acid (CBDVA-C₃); cannabielsoin-type (CBE) cannabinoids, e.g. (5aS,6S,9R,9aR)-cannabielsoin (CBE-C₅), (5aS,6S,9R,9aR)-C₃-cannabielsoin (CBE-C₃), (5aS,6S,9R,9aR)-cannabielsoic acid A (CBEA-C₅ A), (5aS,6S,9R,9aR)-cannabielsoic acid B (CBEA-C₅ B), (5aS,6S,9R,9aR)-C₃-cannabielsoic acid B (CBEA-C₃ B), cannabiglendol-C₃ (OH-iso-HHCV-C₃), dehydrocannabifuran (DCBF-C₅), and cannabifuran (CBF-C₅); cannabigerol-type (CBG) cannabinoids, e.g. cannabigerol ((E)-CBG-C), cannabigerol monomethyl ether ((E)-CBGM-C₅ A), cannabinerolic acid A ((Z)-CBGA-C₅ A), cannabigerovarin ((E)-CBGV-C₃), cannabigerolic acid A ((E)-CBGA-C₅ A), cannabigerolic acid A monomethyl ether ((E)-CBGAM-C₅ A), and cannabigerovarinic acid A ((E)-CBGVA-C₃ A); cannabicyclol-type (CBL) cannabinoids, e.g. (±)-(1aS,3aR,8bR,8cR)-cannabicyclol (CBL-C₅), (±)-(1aS,3aR,8bR,8cR)-cannabicyclolic acid A (CBLA-C₅ A), and (±)-(1aS,3aR,8bR,8cR)-cannabicyclovarin (CBLV-C₃); cannabinol-type (CBN) cannabinoids, e.g. cannabinol (CBN-C₅), cannabinol-C₄ (CBN-C₄), cannabivarin (CBN-C₃), cannabinol-C₂ (CBN-C₂), cannabiorcol (CBN-C₁), cannabinolic acid A (CBNA-C₅ A), and cannabinol methyl ether (CBNM-C₅); cannabinodiol-type (CBND) cannabinoids, e.g. cannabinodiol (CBND-C₅) and cannabinodivarin (CBND-C₃); cannabicitran-type or cannabitriol-type (CBT) cannabinoids, e.g. cannabicitran (CBT-C), (−)-(9R,10R)-trans-cannabitriol ((−)-trans-CBT-C₅), (+)-(9S,10S)-cannabitriol ((+)-trans-CBT-C₅), (±)-(9R,10S/9S,10R)-cannabitriol ((±)-cis-CBT-C₅), (−)-(9R,10R)-trans-10-O-ethyl cannabitriol ((−)-trans-CBT-OEt-C₅), (±)-(9R,10R/9S,10S)-cannabitriol-C₃ ((±)-trans-CBT-C₃), 8,9-dihydroxy-Δ^{6a(10a)}-tetrahydrocannabinol (8,9-Di-OH-CBT-C₅), cannabidiolic acid A cannabitriol ester (CBDA-C₅ 9-OH-CBT-C₅ ester), cannabiripsol (cannabiripsol-C₅), and (−)-6a,7,10a-trihydroxy-Δ⁹-tetrahydrocannabinol ((−)-cannabitetrol); and isocannabinoids, e.g. (−)-Δ⁷-trans-(1R,3R,6r)-isotetrahydrocannabinol, (±)-Δ⁷-1,2-cis-(1R,3R,6S/1S,3S,6R)-isotetrahydrocannabivarin, and (−)-Δ⁷-trans-(1R,3R,6R)-isotetrahydrocannabivarin. It is to be understood that the precise composition of the substantially THC-free composition may vary widely within the scope of the present application; all that is required is that the fluid(s) be substantially free of THC. The substantially THC-free composition will, in most embodiments, generally be provided as a liquid, but substantially THC-free compositions in the solid and gas phases are contemplated and are within the scope of the present invention.

In some embodiments, the substantially THC-free composition may comprise at least one terpene or terpenoid. Non-limiting examples of such terpenes and terpenoids include endo-borneol; δ-carene; bornyl acetate; α-ylangene; α-copaene; aromadendrene; eremophilene; longifolene; β-guaiene; valencene; β-bisabolene; γ-cadinene; β-selinene; neophytadiene; ferruginol; aristolone; β-amyrin; oleanane; ketoursene; α-amyrin; iridoids; iridoid glycosides; steroids, e.g. campesterol, β-sitosterol, γ-sitosterol, stigmasterol, tocopherols, cholesterol, testosterone, cholecalciferol, and ecdysone; hemiterpenoids, e.g. isoprene, prenol, and isovaleric acid; acyclic monoterpenes, e.g. ocimene and myrcenes; monocyclic monoterpenes, e.g. limonene, terpinene, phellandrene, and umbellulone; bicyclic monoterpenes, e.g. pinene a, pinene p, camphene, thujene, sabinene, and carene; acyclic monoterpenoids, e.g. linalool, citronellal, citral, citronellol, geraniol, and geranyl pyrophosphate; monocyclic monoterpenoids, e.g. grapefruit mercaptan, menthol, p-cymene, thymol, perillyl alcohol, and carvacrol; bicyclic monoterpenoids, e.g. camphor, borneol, eucalyptol, halomon, and ascaridole; sesquiterpenoids, e.g. farnesyl pyrophosphate, artemisinin, and bisabolol; diterpenoids, e.g. geranylgeranyl pyrophosphate, gibberellin, retinol, retinal, phytol, taxol, forskolin, aphidicolin, and salvinorin A; sesterterpenoids, e.g. geranylfarnesol; non-steroidal triterpenoids, e.g. saponins, squalene, lanosterol, oleanolic acid, ursolic acid, betulinic acid, and moronic acid); sesquarterpenes and sesquarterpenoids, e.g. ferrugicadiol and tetraprenylcurcumene; carotenes, e.g. α-carotene, β-carotene, γ-carotene, δ-carotene, lycopene, neurosporene, phytofluene, and phytoene; xanthophylls, e.g. canthaxanthin, cryptoxanthin, zeaxanthin, astaxanthin, lutein, and rubixanthin; polyterpenoids; norisoprenoids, e.g. 3-oxo-α-ionol, 7,8-dihydroionone, and precursors thereto; and activated isoprenes, e.g. isopentenyl pyrophosphate (IPP), dimethylallyl pyrophosphate (DMAPP), and precursors thereto.

After providing the substantially THC-free composition, the substantially THC-free composition is dissolved in a suitable solvent to form a substantially THC-free solution. Solvents suitable for use in the practice of the present invention include any bio-compatible liquid in which the substantially THC-free composition is soluble. By way of non-limiting example, the solvent in which the substantially THC-free composition is dissolved may be a bio-compatible organic solvent, preferably a bio-compatible alcohol and even more preferably ethanol.

After providing the substantially THC-free solution, the substantially THC-free solution is applied to a composition of one or more smokable herbs by any suitable means. By way of non-limiting example, the substantially THC-free solution may be applied to the smokable herb composition by any one or more of spraying the substantially THC-free solution onto the smokable herb composition, dipping the smokable herb composition into a vessel of the substantially THC-free solution, soaking the smokable herb composition with the substantially THC-free solution, and any other suitable means. It is to be expressly understood that any means, method, or process for applying the substantially THC-free solution to the smokable herb composition is contemplated as being within the scope of the present invention, so long as such method/process does not introduce carcinogenic, toxic, or otherwise harmful substances into the smokable herb composition.

Any herb or other plant commonly or suitably burned for the purpose of inhalation of the resulting smoke by a user may be provided in the smokable herb compositions of the present invention. By way of non-limiting example, the smokable herb compositions of the present invention may comprise any one or more of Althaea officinalis (marshmallow), Amaranthus dubius, Arctostaphylos uva-ursi (bearberry), Argemone mexicana, plants of the genus Arnica, Artemisia vulgaris (mugwort), plants of the family Asteraceae (chamomile), Brassica oleracea (cabbage), Calea zacatechichi, Canavalia maritima (baybean), plants of the genus Cannabis, Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), plants of the genus Lavandula (lavender), Lactuca virosa (lettuce opium), plants in the family Lamiaceae (mint) including but not limited to plants of the genuses Salvia (sage) and Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), species of the genus Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (both known as wireweed), Silene capensis, Syzygium aromaticum (clove), Tagetes lucida (Mexican tarragon), Tarchonanthus camphoratus, Turnera diffusa (damiana), Tussilago farfara (coltsfoot), species of the genus Verbascum (mullein), and Zornia latifolia (maconha brava).

After the substantially THC-free solution has been applied to the smokable herb composition, the solvent is allowed to evaporate, leaving the substantially THC-free composition applied to the smokable herb composition. Evaporation of the solvent may be passive (i.e. the smokable herb composition with the applied substantially THC-free solution may be exposed to ambient air and allowed to dry naturally) or active (e.g. by heating to drive off the volatile solvent). It is to be understood that the composition of the finished smokable herb composition, i.e. the smokable herb composition with the applied substantially THC-free composition may vary based on the composition of the substantially THC-free composition and the smokable herb composition and the identity and nature of the solvent, and/or may be controlled by controlling various parameters and characteristics of the dissolution, application, and evaporation steps, and that such variations are within the scope of the present application.

Finished smokable herb compositions and products including a substantially THC-free composition as provided by the current invention have many potential uses and applications, for which other smokable herb compositions may not be suitable. As a first non-limiting example, smokable herb compositions and products obtained by the methods and systems of the present application may be used as delivery devices for CBD or other compounds having therapeutic uses, for instance in the treatment of epilepsy or other neurological disorders. As a second non-limiting example, smokable herb compositions and products obtained by the methods and systems of the present application may be used as cigarette substitutes and/or smoking cessation aids. It is to be understood that the above examples are non-limiting, and any application or use of a smokable herb composition that is substantially free of THC is within the scope of the present application.

It is to be expressly understood that the methods described herein may be suitable to infuse any plant material, including but not limited to hemp, blends of herbs, corn silk, etc., with nicotine, at least one terpene, at least one cannabinoid, or a combination or mixture thereof. The resulting infused plant material may be provided in a physical form that enables the infused plant material to be smoked (i.e. combusted and the resulting smoke inhaled by a user) and may have any of a wide variety of uses. In embodiments, such infused plant materials may be useful as smoking cessation aids or apparatuses for use in a smoking cessation method, whereby varying dosages of nicotine, cannabinoids (e.g. CBD), and/or terpenes may be employed to gradually attenuate a user's dependence on nicotine.

The present disclosure, in various aspects, embodiments, and configurations, includes components, methods, processes, systems and/or apparatus substantially as depicted and described herein, including various aspects, embodiments, configurations, sub-combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A smokable product, comprising:
a composition of one or more smokable herbs; and
a substantially THC-free composition.

2. The smokable product of claim 1, wherein the composition of one or more smokable herbs comprises at least one herb selected from the group consisting of Althaea officinalis (marshmallow), Amaranthus dubius, Arctostaphylos uva-ursi (bearberry), Argemone mexicana, plants of the genus Arnica, Artemisia vulgaris (mugwort), plants of the family Asteraceae (chamomile), Brassica oleracea (cabbage), Calea zacatechichi, Canavalia maritima (baybean), plants of the genus Cannabis, Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), plants of the genus Lavandula (lavender), Lactuca virosa (lettuce opium), plants in the family Lamiaceae (mint) including but not limited to plants of the genuses Salvia (sage) and Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), species of the genus Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (both known as wireweed), Silene capensis, Syzygium aromaticum (clove), Tagetes lucida (Mexican tarragon), Tarchonanthus camphoratus, Turnera diffusa (damiana), Tussilago farfara (coltsfoot), species of the genus Verbascum (mullein), and Zornia latifolia (maconha brava).

3. The smokable product of claim 1, wherein the substantially THC-free composition comprises any one or more compounds commonly found in or derived from plants of the genus Cannabis.

4. The smokable product of claim 3, wherein the substantially THC-free composition is, or is derived from, a distillate obtained from plants of the genus Cannabis.

5. The smokable product of claim 1, wherein the substantially THC-free composition comprises at least one cannabinoid selected from the group consisting of a cannabichromene-type (CBC) cannabinoid, a cannabichromanone-type (CBCN) cannabinoid, a cannabidiol-type (CBD) cannabinoid, a cannabielsoin-type (CBE) cannabinoid, a cannabigerol-type (CBG) cannabinoid, a cannabicyclol-type (CBL) cannabinoid, a cannabinol-type (CBN) cannabinoid, a cannabinodiol-type (CBND) cannabinoid, a cannabicitran-type or cannabitriol-type (CBT) cannabinoid, and an isocannabinoid.

6. The smokable product of claim 1, wherein the substantially THC-free composition comprises at least one terpene or terpenoid selected from the group consisting of endo-borneol, δ-carene, bornyl acetate, α-ylangene, α-copaene, aromadendrene, eremophilene, longifolene, β-guaiene, valencene, β-bisabolene, γ-cadinene, β-selinene, neophytadiene, ferruginol, aristolone, β-amyrin, oleanane, ketoursene, α-amyrin, an iridoids, an iridoid glycoside, a steroid, a hemiterpenoid, an acyclic monoterpene, a monocyclic monoterpene, a bicyclic monoterpene, an acyclic monoterpenoid, a monocyclic monoterpenoid, a bicyclic monoterpenoid, a sesquiterpenoid, a diterpenoid, a sesterterpenoid, a non-steroidal triterpenoid, a sesquarterpene, a sesquarterpenoid, a carotene, a xanthophyll, a polyterpenoid, a norisoprenoid, and an activated isoprene.

7. The smokable product of claim 1, comprising no more than 0.3 wt % THC.

8. The smokable product of claim 1, wherein the smokable product is useful as a smoking cessation aid.

9. A method for making a smokable product, comprising:
dissolving a substantially THC-free composition in a liquid solvent to form a substantially THC-free solution;
applying the substantially THC-free solution to a composition of one or more smokable herbs; and
evaporating the liquid solvent.

10. The method of claim 9, wherein the substantially THC-free composition comprises any one or more compounds commonly found in or derived from plants of the genus Cannabis.

11. The method of claim 10, wherein the substantially THC-free composition is, or is derived from, a distillate obtained from plants of the genus Cannabis.

12. The method of claim 9, wherein the substantially THC-free composition comprises at least one cannabinoid selected from the group consisting of a cannabichromene-type (CBC) cannabinoid, a cannabichromanone-type (CBCN) cannabinoid, a cannabidiol-type (CBD) cannabinoid, a cannabielsoin-type (CBE) cannabinoid, a cannabigerol-type (CBG) cannabinoid, a cannabicyclol-type (CBL) cannabinoid, a cannabinol-type (CBN) cannabinoid, a cannabinodiol-type (CBND) cannabinoid, a cannabicitran-type or cannabitriol-type (CBT) cannabinoid, and an isocannabinoid.

13. The method of claim 9, wherein the substantially THC-free composition comprises at least one terpene or terpenoid selected from the group consisting of endo-borneol, δ-carene, bornyl acetate, α-ylangene, α-copaene, aromadendrene, eremophilene, longifolene, β-guaiene, valencene, β-bisabolene, γ-cadinene, β-selinene, neophytadiene, ferruginol, aristolone, β-amyrin, oleanane, ketoursene, α-amyrin, an iridoids, an iridoid glycoside, a steroid, a hemiterpenoid, an acyclic monoterpene, a monocyclic monoterpene, a bicyclic monoterpene, an acyclic monoterpenoid, a monocyclic monoterpenoid, a bicyclic monoterpenoid, a sesquiterpenoid, a diterpenoid, a sesterterpenoid, a non-steroidal triterpenoid, a sesquarterpene, a sesquarterpenoid, a carotene, a xanthophyll, a polyterpenoid, a norisoprenoid, and an activated isoprene.

14. The method of claim 9, wherein the liquid solvent is an organic solvent.

15. The method of claim 14, wherein the organic solvent comprises an alcohol.

16. The method of claim 15, wherein the alcohol is ethanol.

17. The method of claim 9, wherein the composition of one or more smokable herbs comprises at least one herb selected from the group consisting of Althaea officinalis (marshmallow), Amaranthus dubius, Arctostaphylos uva-ursi (bearberry), Argemone mexicana, plants of the genus Arnica, Artemisia vulgaris (mugwort), plants of the family Asteraceae (chamomile), Brassica oleracea (cabbage), Calea zacatechichi, Canavalia maritima (baybean), plants of the genus Cannabis, Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), plants of the genus Lavandula (lavender), Lactuca virosa (lettuce opium), plants in the family Lamiaceae (mint) including but not limited to plants of the genuses Salvia (sage) and Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), species of the genus Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (both known as wireweed), Silene capensis, Syzygium aromaticum (clove), Tagetes lucida (Mexican tarragon), Tarchonanthus camphoratus, Turnera diffusa (damiana), Tussilago farfara (coltsfoot), species of the genus Verbascum (mullein), and Zornia latifolia (maconha brava).

18. The method of claim 9, wherein the smokable product comprises no more than 0.3 wt % THC.

19. The method of claim 9, wherein the smokable product is useful as a smoking cessation aid.

20. A method for aiding a subject in smoking cessation, comprising:
providing to the subject a smoking cessation composition, wherein the smoking cessation composition comprises a plant material infused with at least one of nicotine, a cannabinoid, a terpene, and a terpenoid.
